(19) United States
(12) Patent Application Publication
(10) Pub. No.: US 2023/0207131 A1
(43) Pub. Date: Jun. 29, 2023

(54) WOUND MANAGEMENT SYSTEM FOR PREDICTING AND AVOIDING WOUNDS IN A HEALTHCARE FACILITY

(71) Applicant: MatrixCare, Inc., Bloomington, MN (US)

(72) Inventors: Jessica ROCKNE, West Jordan, UT (US); Vivek KUMAR, Eden Prairie, MN (US); Adhiraj Ganpat PRAJAPATI, St. Paul, MN (US); Robert PRICE, Lockport, IL (US); Kedar Mangesh KADAM, Nova Scotia, MN (US); Timothy James HEEREN, Bloomington, MN (US); Nitin GANDHI, North Liberty, IA (US); Coleen Patrice DANIELSON, Fergus Falls, MN (US)

(21) Appl. No.: 17/562,789

(22) Filed: Dec. 27, 2021

(51) Int. Cl. G16H 50/30; G16H 10/60; G06N 7/00; G16H 50/70

(52) U.S. Cl. CPC G16H 50/30; G06N 7/005; G16H 10/60; G16H 50/70

(57) ABSTRACT

Certain aspects of the present disclosure provide a wound management system and method for predicting and avoiding wounds in a healthcare facility. The method includes collecting data relating to a patient’s health and applying a machine learning model to the data relating to the patient’s health to predict a first probability that the patient will sustain a first wound while the patient is at a first healthcare facility. The method also includes determining an action that reduces the first probability that the patient will sustain the first wound and communicating, to the first healthcare facility, a message indicating that the action should be taken to reduce the first probability that the patient will sustain the first wound while the patient is at the first healthcare facility.

### INTRODUCTION

Aspects of the present disclosure relate to a wound management system for predicting and avoiding wounds in a healthcare facility. Over time, healthcare has become an increasingly demanding field. As the number of patients increases and as the needs of these patients increase, so does the demand and pressure placed on healthcare facilities. Despite best attempts, wounds still occur in healthcare settings. Because the approach to wounds at many healthcare facilities is reactive (e.g., the wounds are treated when they occur), there are many techniques for treating and healing different types of wounds, but the incidences or occurrences of wounds is not necessarily decreasing.

Additionally, patients often relied on word of mouth and anecdotes to assess the likelihood that the patients would sustain wounds while the patients were at certain healthcare facilities. These assessments were often based on incomplete information and bias, which resulted in inaccuracy and did not actually improve the health and well-being of the patient or reduce the incidences or occurrences of wounds.

### SUMMARY

A wound management system and method for predicting and avoiding wounds in a healthcare facility are described herein. According to an embodiment, a method includes collecting data relating to a patient’s health comprising symptoms experienced by the patient and in response to determining that the symptoms experienced by the patient necessitate in-patient care, applying a machine learning model to the data relating to the patient’s health to predict a first probability that the patient will sustain a first wound while the patient is at a first healthcare facility. The method also includes, in response to determining, based on the first probability, that the patient should be treated at the first healthcare facility, determining an action that reduces the first probability that the patient will sustain the first wound and communicating, to the first healthcare facility, a message indicating that the action should be taken to reduce the first probability that the patient will sustain the first wound while the patient is at the first healthcare facility. Other embodiments include an apparatus and a processing system that perform this method. Additional embodiments include a non-transitory computer-readable medium and a computer program product that include instructions that, when executed by a processor, cause the processor to perform this method.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 illustrates an example system.

FIG. 2 illustrates an example wound management system in the system of FIG. 1.

FIG. 3 illustrates example previous patient data in the system of FIG. 1.

FIG. 4 illustrates example health screening data in the system of FIG. 1.

FIG. 5 illustrates an example wound management system in the system of FIG. 1.

FIG. 6 illustrates an example wound management system in the system of FIG. 1.

FIG. 7 illustrates an example wound management system in the system of FIG. 1.

FIG. 8 illustrates an example operation performed in the system of FIG. 1.

FIG. 9 is a flowchart of an example method performed in the system of FIG. 1.

FIG. 10 is a flowchart of an example method performed in the system of FIG. 1.

FIG. 11 is a flowchart of an example method performed in the system of FIG. 1.

FIG. 12 illustrates an example device in the system of FIG. 1.

FIG. 13 illustrates an example device in the system of FIG. 1.

FIG. 14 illustrates an example device in the system of FIG. 1.

FIG. 15 illustrates an example device in the system of FIG. 1.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer readable mediums for predicting and avoiding wounds in a healthcare facility. Specifically, this disclosure describes a wound management system that uses machine learning to predict whether a patient is likely to sustain different wounds at different healthcare facilities based on information about the patient. For example, the wound management system may predict that a patient is more likely to sustain particular wounds at one healthcare facility relative to another healthcare facility. In response, the wound management system may recommend that the patient visit one healthcare facility over the other. As another example, the wound management system may predict that a patient has an unacceptably high likelihood of sustaining a particular wound at a healthcare facility. In response, the wound management system may recommend that certain remedial actions be taken by the healthcare facility to prevent the wound from occurring. In this manner, the wound management system provides a proactive approach towards wound treatment, which improves the health and well-being of the patient, in certain embodiments.

### Example Systems and Methods

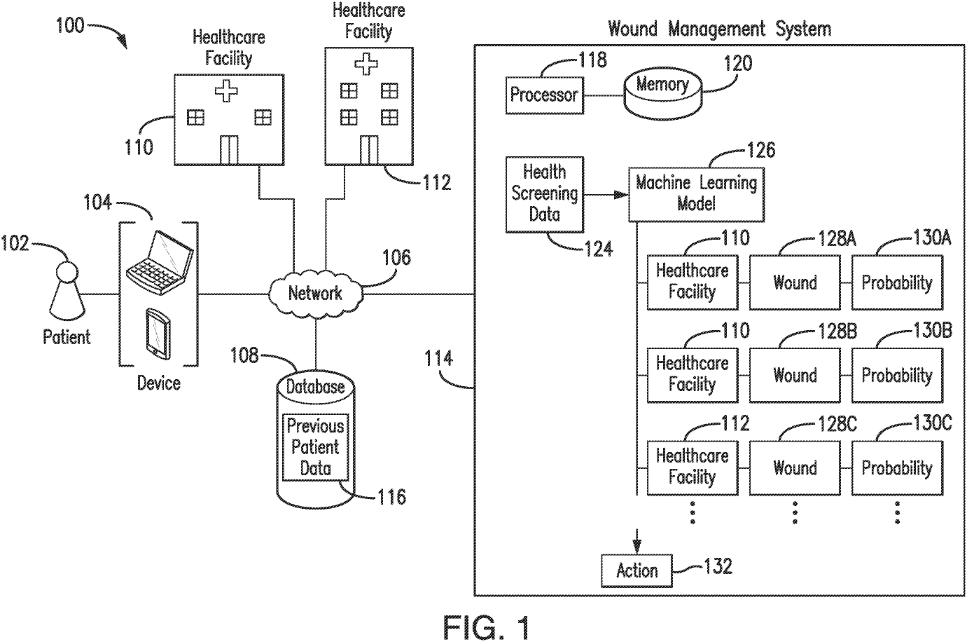

FIG. 1 illustrates an example system 100. As seen in FIG. 1, the system 100 includes one or more devices 104, a network 106, a database 108, healthcare facilities 110 and 112, and a wound management system 114. Generally, the system 100 uses machine learning to determine likelihoods of a patient 102 sustaining certain wounds while at the healthcare facilities 110 and 112. The system 100 may then recommend that the patient 102 visit a particular healthcare facility for a reduced likelihood that the patient 102 will sustain a particular wound. Additionally, the system 100 may recommend actions to the healthcare facility that will reduce the probability that the patient 102 will sustain a particular wound while at that healthcare facility. In this manner, the system 100 takes a proactive approach to wound treatment, which reduces the incidences or occurrences of wounds and improves the health and well-being of the patient 102 in particular embodiments.

Specifically, patients 102 often relied on word of mouth and anecdotes to assess the likelihood that the patients 102 would sustain wounds while the patients 102 were at certain healthcare facilities. For example, pregnant mothers would choose certain healthcare facilities for delivery based on stories or accounts of positive or negative experiences that other mothers had at these healthcare facilities. As another example, patients 102 with certain health conditions would choose certain healthcare facilities because these healthcare facilities had a reputation for treating these conditions, but the patients 102 merely assumed that they would not sustain wounds while they were at these healthcare facilities. Therefore, the patients 102 were not performing an accurate assessment of their likelihood of sustaining wounds, which did not improve the health and well-being of the patients 102. Additionally, the healthcare facilities did not assess the likelihood of the patients 102 sustaining wounds. Rather, the healthcare facilities merely treated wounds as they were sustained. Therefore, the incidences and occurrences of wounds was not decreasing.

The wound management system 114, on the other hand, uses machine learning to assess the likelihood of a patient 102 sustaining a wound at certain healthcare facilities (e.g., healthcare facilities 110 and 112). Specifically, the wound management system 114 uses machine learning to perform a complete analysis on the patient’s 102 information by comparing that information with the information of previous patients who sustained wounds at the healthcare facilities. As a result, the wound management system 114 provides a more accurate prediction of the likelihood that the patient 102 will sustain a wound at the healthcare facilities, in certain embodiments. The wound management system 114 also provides recommendations to the patient 102 (e.g., a healthcare facility recommendation) and to the healthcare facilities (e.g., a treatment or remedy plan recommendation) based on these more accurate predictions, which effects a particular treatment or prophylaxis for preventing or reducing the likelihood of sustaining wounds at the healthcare facilities.

A patient 102 may use the device 104 to provide information about the patient 102. This information may then be used to predict the likelihood that the patient 102 will sustain particular wounds at certain healthcare facilities. The patient 102 may provide the information when the patient 102 is experiencing symptoms and is determining whether to visit a healthcare facility. The device 104 may execute an application that the patient 102 uses to provide the information about the patient 102. For example, the patient 102 may provide information that identifies the patient 102 such as a name and address of the patient 102. Additionally, the patient 102 may explain the symptoms experienced by the patient 102. After the device 104 has collected the information from the patient 102, the device 104 may communicate that information to the wound management system 114 for processing. After the wound management system 114 has analyzed that information, the wound management system 114 may communicate a message back to the device 104 that includes recommendations for the patient 102. For example, the message may recommend a particular healthcare facility for the patient 102. The patient 102 may read the message and follow the recommendations in the message, which may reduce the likelihood that the patient 102 will sustain particular wounds while at the healthcare facility.

The device 104 is any suitable device for communicating with components of the system 100 over the network 106. As an example and not by way of limitation, the device 104 may be a computer, a laptop, a wireless or cellular telephone, an electronic notebook, a personal digital assistant, a tablet, or any other device capable of receiving, processing, storing, or communicating information with other components of the system 100. The device 104 may be a wearable device such as a virtual reality or augmented reality headset, a smart watch, or smart glasses. The device 104 may also include a user interface, such as a display, a microphone, keypad, or other appropriate terminal equipment usable by the patient 102. The device 104 may include a hardware processor, memory, or circuitry configured to perform any of the functions or actions of the device 104 described herein. For example, a software application designed using software code may be stored in the memory and executed by the processor to perform the functions of the device 104.

The network 106 is any suitable network operable to facilitate communication between the components of the system 100. The network 106 may include any interconnecting system capable of transmitting audio, video, signals, data, messages, or any combination of the preceding. The network 106 may include all or a portion of a public switched telephone network (PSTN), a public or private data network, a local area network (LAN), a metropolitan area network (MAN), a wide area network (WAN), a local, regional, or global communication or computer network, such as the Internet, a wireline or wireless network, an enterprise intranet, or any other suitable communication link, including combinations thereof, operable to facilitate communication between the components.

The database 108 stores previous patient data 116. The previous patient data 116 includes information about patients and the wounds that they may have sustained while at the healthcare facilities 110 and 112. The previous patient data 116 may be recorded or logged by the healthcare facilities 110 and 112 while treating patients at these healthcare facilities 110 and 112. As a result, the previous patient data 116 may include information that identifies these patients such as names and addresses. Additionally, the previous patient data 116 may include a record of the wounds sustained by the patients as well as the treatments and remedies that the healthcare facilities 110 and 112 used for these wounds. The previous patient data 116 may be used by the wound management system 114 to train a machine learning model to predict the likelihood that the patient 102 will sustain particular wounds while visiting the healthcare facilities 110 and 112.

The healthcare facilities 110 and 112 may be proximate to the patient 102. The patient 102 may visit these healthcare facilities 110 and 112 when the patient 102 is experiencing symptoms. The device 104 may recommend which of the healthcare facilities 110 or 112 that the patient 102 should visit based on messages received from the wound management system 114. Specifically, the wound management system 114 may predict likelihoods that the patient 102 will sustain certain wounds while visiting these healthcare facilities 110 or 112. Based on these predicted probabilities, the wound management system 114 may recommend that the patient 102 visit a particular healthcare facility 110 or 112. Additionally, the wound management system 114 may recommend a treatment plan or course of action to the recommended healthcare facility 110 or 112 so that the healthcare facility 110 or 112 may reduce the likelihood or probability that the patient 102 will sustain particular wounds while the patient 102 is at the healthcare facility 110 or 112. The healthcare facility 110 or 112 may then implement that treatment plan or course of action while treating the patient 102 to reduce the chances that the patient 102 will sustain particular wounds. In this manner the healthcare facilities 110 or 112 improve the health and wellbeing of the patient 102 while reducing the incidences or occurrences of wounds sustained at the healthcare facilities 110 or 112 in particular embodiments.

The wound management system 114 applies machine learning to information provided by the patient 102 to predict likelihoods or probabilities that the patient 102 will sustain particular wounds while visiting the healthcare facilities 110 or 112. Additionally, the wound management system 114 may provide recommendations to the patient 102 and to the healthcare facilities 110 or 112 that reduce the likelihood that the patient 102 will sustain particular wounds. As a result, the wound management system 114 takes a proactive approach to treating wounds and reduces the likelihood that the patient 102 will sustain particular wounds while at the healthcare facilities 110 or 112 in certain embodiments. As seen in FIG. 1, the wound management system 114 includes a processor 118 and a memory 120, which perform the functions or actions of the wound management system 114 described herein. The wound management system 114 may be a computer system (e.g., a server separate from the device 104 of the patient 102). In some embodiments, the wound management system 114 may be embodied within the device 104. In these embodiments, the processor 118 and the memory 120 of the wound management system 114 may be a processor or a memory within the device 104.

The processor 118 is any electronic circuitry, including, but not limited to one or a combination of microprocessors, microcontrollers, application specific integrated circuits (ASIC), application specific instruction set processor (ASIP), and/or state machines, that communicatively couples to memory 120 and controls the operation of the wound management system 114. The processor 118 may be 8-bit, 16-bit, 32-bit, 64-bit or of any other suitable architecture. The processor 118 may include an arithmetic logic unit (ALU) for performing arithmetic and logic operations, processor registers that supply operands to the ALU and store the results of ALU operations, and a control unit that fetches instructions from memory and executes them by directing the coordinated operations of the ALU, registers and other components. The processor 118 may include other hardware that operates software to control and process information. The processor 118 executes software stored on the memory 120 to perform any of the functions described herein. The processor 118 controls the operation and administration of the wound management system 114 by processing information (e.g., information received from the devices 104, network 106, and memory 120). The processor 118 is not limited to a single processing device and may encompass multiple processing devices.

The memory 120 may store, either permanently or temporarily, data, operational software, or other information for the processor 118. The memory 120 may include any one or a combination of volatile or non-volatile local or remote devices suitable for storing information. For example, the memory 120 may include random access memory (RAM), read only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory 120, a disk, a CD, or a flash drive. In particular embodiments, the software may include an application executable by the processor 118 to perform one or more of the functions described herein.

The wound management system 114 receives health screening data 124 from the device 104. The patient 102 may provide the health screening data 124 using the device 104. The health screening data 124 may include information about the patient 102. For example, the health screening data 124 may include information that identifies the patient 102, such as a name and address. The health screening data 124 may also include medical information of the patient 102, such as symptoms experienced by the patient 102, allergies and vaccinations of the patient 102, previous hospitalizations and operations for the patient 102, and medical histories of family members of the patient 102. Additionally, the health screening data 124 may include information about the lifestyle or habits of the patient 102, such as the home condition or work condition of the patient 102. Additionally the health screening data 124 may indicate events or activities in which the patient 102 engages. The health screening data 124 may also indicate whether the patient 102 smokes, drinks, wears a seatbelt, or uses recreational drugs. The patient 102 may provide the health screening data 124 prior to visiting a healthcare facility 110 or 112 or while checking into the healthcare facility 110 or 112. In some embodiments, the health screening data 124 may be electronic medical records of the patient 102 that are provided by the patient 102 or stored and provided by the healthcare facilities 110 and 112. For example, the patient 102 may instruct the healthcare facilities 110 and 112 to provide the electronic medical records to the wound management system 114.

The wound management system 114 applies a machine learning model 126 to the health screening data 124 to predict likelihoods that the patient 102 will sustain particular wounds while visiting the healthcare facility 110 or the healthcare facility 112. In some embodiments, the wound management system 114 applies the machine learning model 126 to the health screening data 124 in response to a trigger condition being satisfied. For example, the wound management system 114 may review the health screening data 124 to determine whether the patient 102 needs in-patient care. Additionally, the wound management system 114 may apply the machine learning model 126 to the health screening data 124 in response to a determination that the healthcare facilities 110 or 112 near the patient 102 have been flagged for previous instances of wounds sustained by other patients while visiting those healthcare facilities 110 or 112 or in response to a determination that the patient 102 has been flagged for previously sustaining wounds while visiting healthcare facilities. When one or more of these trigger conditions have been satisfied, the wound management system 114 applies the machine learning model 126 to the health screening data 124 to predict likelihoods that the patient 102 will sustain particular wounds while visiting the healthcare facilities 110 or 112.

The wound management system 114 may implement supervised machine learning techniques, unsupervised machine learning techniques, or a combination of supervised and unsupervised learning techniques. In an embodiment, a user or an administrator selects the machine learning model to apply based on knowledge or analysis of the health screening data 124 or the previous patient data 116. For example, the wound management system 114 may begin by applying logistic regression to the health screening data 124 or the previous patient data 116. After that analysis is complete, the user or administrator may select another machine learning model to apply that the user or administrator is more suitable for the data. In some embodiments, the wound management system 114 analyzes the health screening data 124 or the previous patient data 116 and determines a machine learning model to apply to the data based on that analysis. Whether supervised or unsupervised techniques are used, the wound management system 114 may convert the health screening data 124 or the previous patient data 116 to a numerical format, and based on an initial data analysis, data transformation techniques may be chosen (e.g., by the wound management system 114 or by a user or administrator).

In the example of FIG. 1, the wound management system 114 applies the machine learning model 126 to the health screening data 124 to determine probabilities 130 that the patient 102 will sustain particular wounds 128 while visiting one of the healthcare facilities 110 or 112. Specifically, the wound management system 114 determines that the patient 102 has a probability 130A of sustaining a wound 128A while visiting the healthcare facility 110. Additionally, the wound management system 114 determines that the patient 102 has a probability 130B of sustaining a wound 128B while visiting the healthcare facility 110. The wound management system 114 also determines that the patient 102 has a probability 130C of sustaining wound 128C while visiting the healthcare facility 112.

The wound management system 114 then uses these predicted wounds 128 and probabilities 130 to determine remedial actions 132 that may be taken to reduce these probabilities 130. For example, the wound management system 114 may query a database or repository of remedial actions 132 using the predicted wounds 128 and probabilities 130 to determine the remedial actions 132. The wound management system 114 may communicate these remedial actions 132 to the patient 102 or to the healthcare facilities 110 or 112 to alert the patient 102 and the healthcare facilities 110 or 112 of the actions 132 that may be taken to reduce the probabilities 130 that the patient 102 will sustain particular wounds 128 while visiting these healthcare facilities 110 or 112. In this manner, the wound management system 114 reduces the likelihood that the patient 102 will sustain particular wounds while visiting these healthcare facilities 110 or 112, which improves the health and safety of the patient 102 and reduces the incidences and occurrences of wounds in particular embodiments.

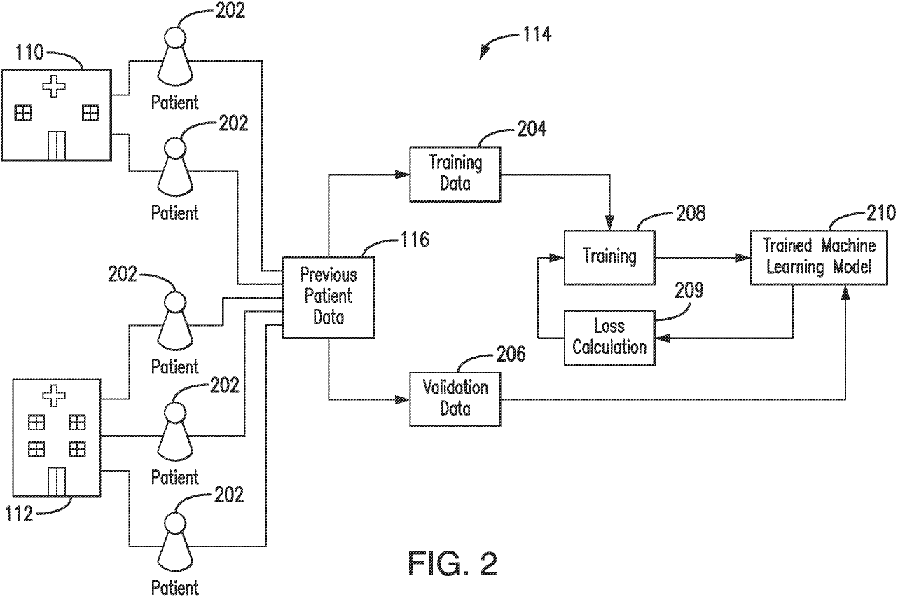

FIG. 2 illustrates an example wound management system 114 in the system 100 of FIG. 1. Generally, FIG. 2 shows the wound management system 114 training a machine learning model using the previous patient data 116. It is contemplated that another computer system separate from the wound management system 114 may train the machine learning model and then deploy the trained machine learning model to the wound management system 114.

As discussed previously, the previous patient data 116 is recorded and stored by the healthcare facilities 110 and 112 as the healthcare facilities 110 and 112 treated previous patients 202 for wounds sustained by those previous patients 202 while visiting the healthcare facilities 110 and 112. The previous patient data 116 may include information that identifies these previous patients 202, including names and addresses. Additionally, the previous patient data 116 may include information that identifies the healthcare facilities 110 and 112, such as the names and addresses of the healthcare facilities 110 and 112. The previous patient data 116 may also include information about the wounds sustained by the previous patients 202. For example, the previous patient data 116 may identify those wounds and include the dates on which those wounds were sustained. The healthcare facilities 110 and 112 record this information and store the previous patient data 116 into the database 108 for later use by the wound management system 114.

The wound management system 114 retrieves the previous patient data 116 from the database 108 and uses the previous patient data 116 to train a machine learning model to predict the likelihood or probability that a patient 102 will sustain a particular wound while visiting the healthcare facility 110 or the healthcare facility 112. In certain embodiments, the wound management system 114 splits or divides the previous patient data 116 into training data 204 and validation data 206. Generally, the wound management system 114 uses the training data 204 to train the machine learning model and then uses the validation data 206 to test the loss or accuracy of the machine learning model.

The wound management system 114 may divide the previous patient data 116 into the training data 204 and the validation data 206 in any suitable manner. For example, the wound management system 114 may divide the previous patient data 116 randomly into the training data 204 and the validation data 206. As another example, the wound management system 114 may select datapoints from the previous patient data 116 that are the most different from each other to form the training data 204. In some embodiments, the wound management system 114 clusters the datapoints in the previous patient data 116 so that the datapoints that are most similar to each other are assigned to the same cluster. The wound management system 114 then selects datapoints from different clusters to form the training data 204. As a result, the training data 204 includes a diverse set of datapoints, which improves the robustness and accuracy of the machine learning model when the machine learning model is trained using the training data 204. After forming the training data 204, the remaining datapoints in the previous patient data 116 may be used to form the validation data 206.

The wound management system 114 uses the training data 204 to train a machine learning model during a training step 208. During the training step 208, the wound management system 114 uses the machine learning model to analyze the datapoints in the training data 204. The machine learning model detects patterns or trends in the datapoints of the training data 204 and learns the particular wounds that correspond or result from these detected patterns or trends. In this manner, the machine learning model is trained to predict the likelihood of certain wounds being sustained when certain patterns or trends are detected.

After the training step 208, the wound management system 114 validates the trained machine learning model 210 using the validation data 206. Generally, the wound management system 114 uses the validation data 206 to determine an accuracy or a loss of the trained machine learning model 210. For example, the wound management system 114 may apply the trained machine learning model 210 to the validation data 206 to detect patterns or trends in the validation data 206 and to predict the likelihood of particular wounds being sustained based on those detected patterns and trends. The wound management system 114 then compares the predicted likelihoods with the actual wounds sustained indicated by the validation data 206 in a loss calculation step 209. Using this comparison, the wound management system 114 determines a loss or accuracy of the trained machine learning model 210. If the loss or accuracy of the trained machine learning model 210 is not at an acceptable level, the wound management system 114 may perform another round of training. If the loss or accuracy of the trained machine learning model 210 is at an acceptable level, the wound management system 114 may conclude training.

In this manner, the machine learning model is trained using data about the healthcare facilities 110 and 112 to predict the likelihood that a patient 102 will sustain a wound while the patient 102 is at these healthcare facilities 110 and 112. Specifically, the machine learning model is trained (i) to detect patterns or trends in data about previous patients 202 that visited these healthcare facilities 110 and 112 and (ii) to learn whether these patterns or trends resulted in certain wounds developing.

After the machine learning model is trained, the wound management system 114 deploys the trained machine learning model 210 to be used on health screening data 124 provided by patients 102. The wound management system 114 may deploy the trained machine learning model 210 to be used by the wound management system 114 itself, or the wound management system 114 may deploy the trained machine learning model 210 to other components of the system 100, such as the device 104 of the patient 102. If the trained machine learning model 210 is deployed to the device 104 of the patient 102, then the device 104 may effectively serve as the wound management system 114.

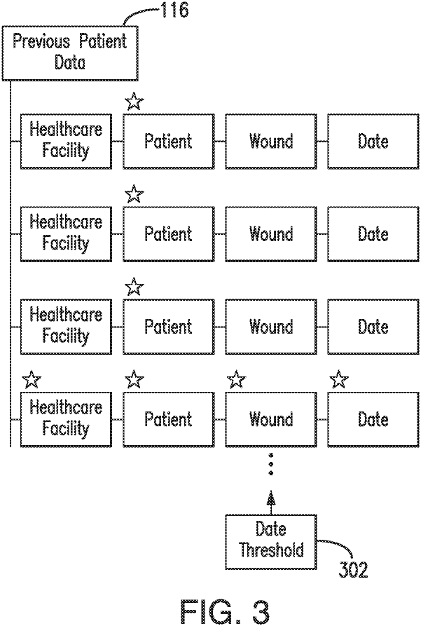

FIG. 3 illustrates example previous patient data 116 in the system 100 of FIG. 1. As discussed previously the previous patient data 116 is recorded or logged by the healthcare facilities 110 and 112 as the healthcare facilities 110 and 112 treated the wounds that the previous patients 202 sustained while visiting the healthcare facilities 110 or 112. As seen in FIG. 3, the previous patient data 116 includes information that identifies the healthcare facility, such as a name or address of the healthcare facility. Additionally, the previous patient data 116 includes information that identifies the previous patient 202, such as a name or address of the previous patient 202. The previous patient data 116 also includes information about the wounds sustained by the previous patients 202. For example, the previous patient data 116 identifies the wounds sustained by the previous patients 202 and the dates on which those previous wounds were sustained. Using this information, the wound management system 114 trains a machine learning model to predict the likelihood that a patient 102 will sustain particular wounds while visiting the healthcare facility 110 or the healthcare facility 112.

In certain embodiments, the wound management system 114 may exclude or remove certain information from the previous patient data 116 before using the previous patient data 116 to train the machine learning model 126. For example, the wound management system 114 may remove information that identifies the previous patients 202 (identified by an ‘*’), such as the names or addresses of the previous patients 202. As a result, the wound management system 114 protects the privacy and security of the previous patients 202. As another example, the wound management system 114 may compare the dates on which the wounds were sustained to a date threshold 302. The wound management system 114 may then remove datapoints (identified by an ‘*’) from the patient data 116 if the date of the wound for that data point is older than the date threshold 302. In this manner, the wound management system 114 trains the machine learning model 126 using newer data as opposed to outdated data, which may improve the accuracy of the machine learning model 126 in certain embodiments.

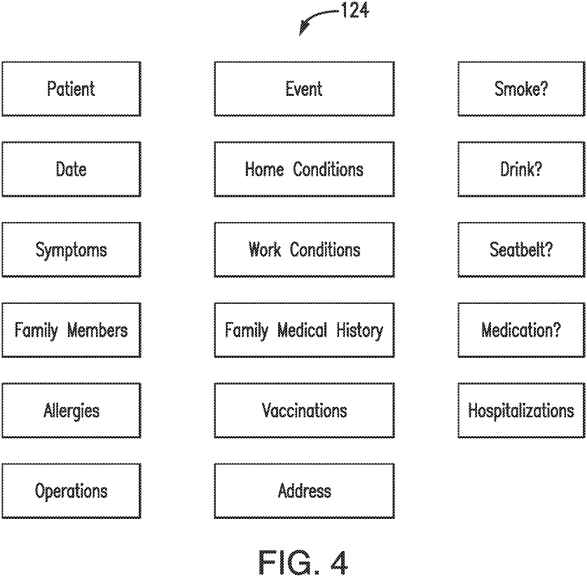

FIG. 4 illustrates example health screening data 124 in the system 100 of FIG. 1. As discussed previously, the patient 102 may use the device 104 to provide the health screening data 124. The device 104 may communicate the health screening data 124 to the wound management system 114, so that the wound management system 114 may apply a machine learning model 126 to the health screening data 124 to predict the likelihoods that the patient 102 will sustain particular wounds while visiting the healthcare facilities 110 or 112.

As seen in FIG. 4, the health screening data 124 includes information about the patient 102. The health screening data 124 may include information that identifies the patient 102, such as a name or address of the patient 102. Additionally, the health screening data 124 may include medical information for the patient 102, such as symptoms experienced by the patient 102 or allergies of the patient 102. The health screening data 124 may include vaccinations or medications taken by the patient 102. The health screening data may also include previous hospitalizations or operations of the patient 102. The health screening data 124 may also include information about the lifestyle or habits of the patient 102. For example, the health screening data 124 may include information about the home conditions or work conditions of the patient 102. The health screening data 124 may also include information about events or activities in which the patient 102 engages. Additionally, the health screening data 124 may include indications of whether the patient 102 smokes, drinks, wears a seatbelt, or uses recreational drugs.

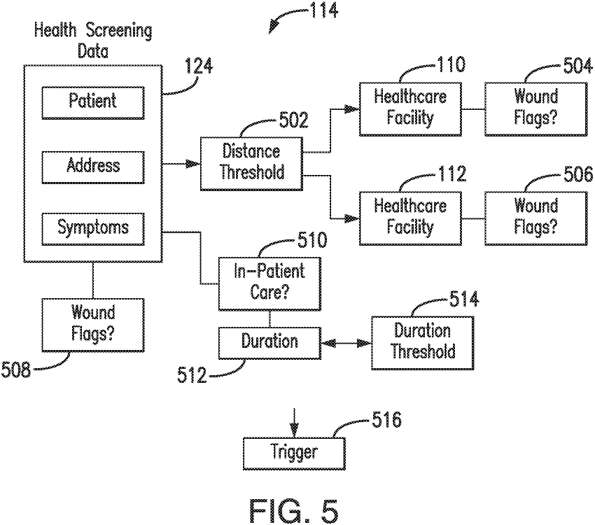

FIG. 5 illustrates an example wound management system 114 in the system 100 of FIG. 1. Generally, FIG. 5 shows the wound management system 114 determining whether trigger conditions have been satisfied for applying the machine learning model 126 to the health screening data 124.

The wound management system 114 receives the health screening data 124 from a patient 102. In the example of FIG. 5, the health screening data 124 includes information that identifies the patient 102, such as a name and address of the patient 102. Additionally, the health screening data 124 includes symptoms experienced by the patient 102. Using this information, the wound management system 114 determines whether certain trigger conditions have been met for applying the machine learning model 126 to the health screening data 124.

The wound management system 114 may first determine the healthcare facilities 110 and 112 that are near the patient 102 based on the address of the patient 102 in the health screening data 124. For example, the wound management system 114 may determine a distance between the address of the patient 102 and the addresses of the healthcare facilities 110 and 112. The wound management system 114 compares these distances to a distance threshold 502. The distance threshold 502 may be set using any suitable manner. For example, the distance threshold 502 may be set by the patient 102, or the distance threshold 502 may be inferred by the wound management system 114 from the health screening data 124 or the previous patient data 116 (e.g., by setting the distance threshold 502 to be an average distance that previous patients 202 traveled to reach a healthcare facility).

If the distance between the patient 102 and a healthcare facility 110 or 112 is below the distance threshold 502, then the wound management system 114 determines that the healthcare facility 110 or 112 is a candidate for the patient 102 to visit. If the distance between a healthcare facility 110 or 112 and the patient 102 exceeds the distance threshold 502, then the wound management system 114 determines that the healthcare facility 110 or 112 is not a candidate for the patient 102 to visit. In the example of FIG. 5, the wound management system 114 determines that the healthcare facilities 110 and 112 are candidates for the patient 102 to visit.

In some embodiments, the wound management system 114 may also consider the patient’s 102 health insurance plan coverage to determine the healthcare facility candidates. For example, the wound management system 114 may exclude certain healthcare facilities from being candidates if the wound management system 114 determines from the health screening data 124 that the patient’s 102 health insurance does not cover services rendered by those healthcare facilities.

The wound management system 114 then determines whether the healthcare facilities 110 and 112 have been previously flagged for patients 202 sustaining wounds while visiting those healthcare facilities 110 or 112. The wound management system 114 may have previously flagged these healthcare facilities 110 or 112 based on information provided by the healthcare facilities 110 and 112 in the previous patient data 116 stored in the database 108. The wound management system 114 determines whether a wound flag 504 has been set for the healthcare facility 110 and whether a wound flag 506 has been set for the healthcare facility 112. If a wound flag is set for a particular healthcare facility, the wound management system 114 may determine that a triggered condition has been satisfied and that the machine learning model 126 should be applied to the health screening data 124 to determine the likelihood that the patient 102 will sustain a wound at the particular healthcare facility.

The wound management system 114 may also determine whether the patient 102 is flagged for previously sustaining wounds while visiting a healthcare facility. The wound management system 114 may have flagged the patient 102 based on information provided by the healthcare facilities in the previous patient data 116. The wound management system 114 determines whether a wound flag 508 has been set for the patient 102. If the wound flag 508 has been set, the wound management system 114 may determine that the machine learning model 126 should be applied to the health screening data 124 to determine the likelihood that the patient 102 will sustain particular wounds while visiting the healthcare facility.

The wound management system 114 may also determine based on the symptoms in the health screening data 124 whether in-patient care is needed in a step 510. For example, the wound management system 114 may analyze the symptoms in the health screening data 124 to determine their severity and their impact on the health of the patient 102. If the severity or impact on the patient 102 is high, the wound management system 114 may determine that in-patient care is needed in step 510. In some embodiments, the wound management system 114 may also determine a duration 512 of the in-patient care. The wound management system 114 compares the determined duration 512 with a duration threshold 514 to determine whether a triggered condition has been met. If the duration 512 exceeds the duration threshold 514, the wound management system 114 may determine that a trigger condition has been met and that the machine learning model 126 should be applied to the health screening data 124 to predict the likelihood that the patient 102 will sustain the particular wounds while visiting the healthcare facility.

The wound management system 114 uses one or more of these various checks to determine whether a trigger 516 is triggered. For example, the trigger condition may be satisfied (i) if in-patient care of a duration 512 that exceeds the duration threshold 514 is needed and (ii) if either the patient 102 or any of the healthcare facilities 110 and 112 have been flagged for previous wounds. If the trigger condition for the trigger 516 is met, the wound management system 114 applies the machine learning model 126 to the health screening data 124. If the trigger condition of the trigger 516 is not met, the wound management system 114 may not apply the machine learning model 126 to the health screening data 124.

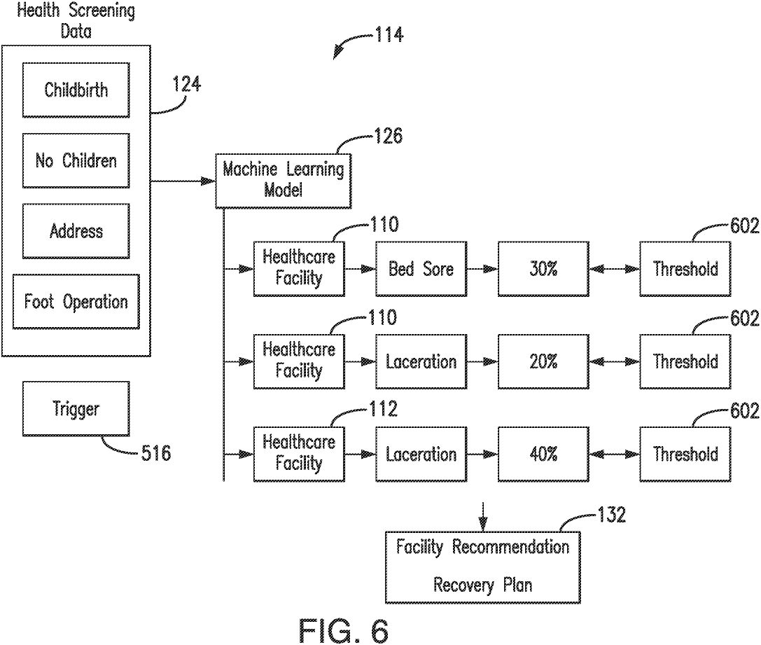

FIG. 6 illustrates an example wound management system 114 in the system 100 of FIG. 1. Generally, FIG. 6 shows the wound management system 114 applying the machine learning model 126 to health screening data 124.

The wound management system 114 receives the health screening data 124 from a patient 102. In the example of FIG. 6, the health screening data 124 indicates that the patient 102 is experiencing symptoms associated with childbirth or labor. The health screening data 124 also indicates that the patient 102 has no children, which suggests that this may be a first time for the patient 102 giving birth. Additionally, the health screening data 124 includes an address of the patient 102 and an indication that the patient 102 had previously had a foot operation. The wound management system 114 may perform each of the checks discussed in FIG. 5 to determine whether a trigger condition of a trigger 516 is met. For example, the wound management system 114 may determine the healthcare facilities 110 and 112 near the patient 102. Additionally, the wound management system 114 may determine that the symptoms experienced by the patient 102 would require in-patient care or that the healthcare facilities 110 and 112 have been previously flagged for patients 202 sustaining particular wounds while visiting those healthcare facilities 110 and 112. In response to the triggered condition being met, the wound management system 114 applies the machine learning model 126 to the health screening data 124.

By applying the machine learning model 126 to the health screening data 124, the wound management system 114 predicts the likelihoods that the patient 102 will sustain particular wounds while visiting the healthcare facilities 110 and 112. In the example of FIG. 6, the machine learning model 126 determines that if the patient 102 were to visit the healthcare facility 110, then the patient 102 would have a 30% chance of developing bedsores and a 20% chance of sustaining lacerations (e.g., perineal lacerations). Additionally, the machine learning model 126 predicts that if the patient 102 were to visit the healthcare facility 112, then the patient 102 would have a 40% chance of sustaining lacerations.

The wound management system 114 compares the predicted likelihoods with a threshold 602 to determine whether remedial action 132 is needed. For example, if a predicted likelihood exceeds the threshold 602, then the wound management system 114 may determine that remedial action 132 is needed. If a predicted likelihood does not exceed the threshold 602, then the wound management system 114 may determine that remedial action 132 is not needed.

In the example of FIG. 6, the wound management system 114 determines a particular facility recommendation and recovery plan as part of the remedial action 132. The wound management system 114 may compare the predicted likelihoods against the threshold 602 to determine which healthcare facility 110 or 112 is better suited for the patient 102. For example, the wound management system 114 may determine, based on the predicted likelihoods, that the healthcare facility 110 is better suited for the childbirth symptoms experienced by the patient 102 due to the healthcare facility 112 having a 40% chance of perineal lacerations, a severe wound. Thus, the action 132 may include a recommendation that the patient 102 visit the healthcare facility 110. Additionally, because the patient 102 had a 30% chance of sustaining bedsores while visiting the healthcare facility 110, the wound management system 114 may determine a recovery plan as part of the action 132 that reduces the likelihood of sustaining bedsores. For example, the wound management system 114 may determine that the patient 102 should be moved regularly and that the bedding for the patient 102 should be changed regularly to reduce the likelihood that the patient 102 will sustain bedsores while visiting the healthcare facility 110. Thus, the wound management system can identify an action (e.g. a medication, a medical procedure, or another action) to reduce the likelihood that the patient will sustain a wound (e.g., bedsores, sutures, abrasions, lesions, or any other wound). As one example, a patient could be provided with medical treatment (e.g., bandaging, a surgical procedure, a particular medication, or any other suitable treatment), to reduce the likelihood of sustaining a wound. For example, a bedsore, suture, abrasion, or lesion could be identified as less likely to occur if the patient receives identified medication, bandaging, or another medical procedure. The wound management system 114 may then communicate the action 132 to the device 104 and to the healthcare facility 110. As a result, the likelihood that the patient 102 will sustain particular wounds while visiting the healthcare facility 110 are reduced in certain embodiments.

In some embodiments, the wound management system 114 first determines which healthcare facility should be recommended to the patient 102 and then the wound management system 114 determines the remedial action 132 in response to the determination of the healthcare facility. For example, if the wound management system 114 determines that the healthcare facility 110 should treat a patient 102 but that patients have an unacceptably high likelihood of developing bedsores while the patients are at the healthcare facility 110, the wound management system 114 may determine the remedial action 132 for avoiding or preventing bedsores should be communicated to the healthcare facility 110.

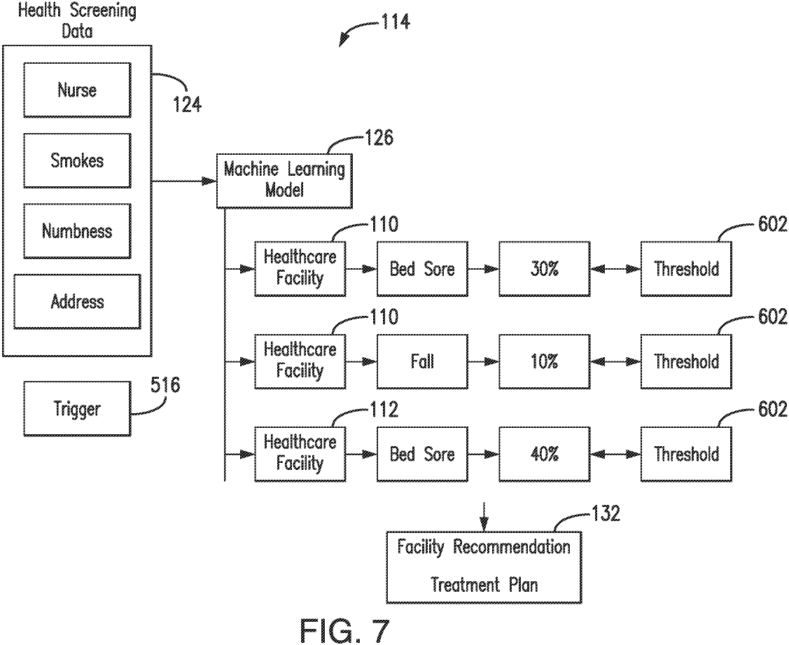

FIG. 7 illustrates an example wound management system 114 in the system 100 of FIG. 1. Generally, FIG. 7 shows the wound management system 114 applying the machine learning model 126 to health screening data 124 of a patient.

In the example of FIG. 7, the health screening data 124 indicates that the patient 102 is a nurse who is experiencing numbness. Additionally the health screening data 124 includes the address of the patient 102 and indicates that the patient 102 smokes. The wound management system 114 performs one or more of the checks described in FIG. 5 to determine whether a trigger condition of a trigger 506 is satisfied. If the trigger condition is satisfied, the wound management system 114 applies the machine learning model 126 to the health screening data 124.

The wound management system 114 applies the machine learning model 126 to the health screening data 124 to predict the likelihoods that the patient 102 will sustain particular wounds while visiting the healthcare facilities 110 or 112. In the example of FIG. 7, the machine learning model 126 predicts that the patient 102 has a 30% chance of sustaining bedsores and a 10% chance of sustaining a wound from a fall if the patient 102 were to visit the healthcare facility 110. Additionally, the machine learning model 126 predicts that the patient 102 has a 40% chance of sustaining bedsores if the patient 102 were to visit the healthcare facility 112.

The wound management system 114 compares the predicted likelihoods with a threshold 602 to determine whether remedial action 132 is needed. In the example of FIG. 7, the wound management system 114 determines that remedial action 132 is needed. The wound management system 114 determines a facility recommendation based on the predicted likelihoods and includes the facility recommendation in the action 132. The wound management system 114 may recommend the healthcare facility 110 due to its lower likelihood of developing bedsores and its low risk of fall wounds. Additionally, the wound management system 114 determines a treatment plan based on the predicted wounds and includes the treatment plan in the action 132. The wound management system 114 then communicates the action 132 to the patient 102 and the healthcare facility 110 to reduce the likelihood that the patient 102 will sustain certain wounds while visiting those healthcare facility 110.

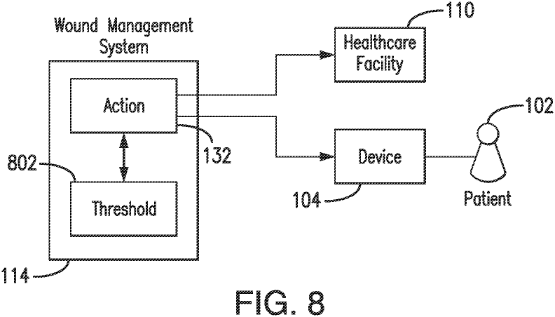

FIG. 8 illustrates an example operation performed in the system 100 of FIG. 1. Generally, FIG. 8 shows the wound management system 114 sending the determined actions 132 to the patient 102 and the healthcare facility 110.

As discussed previously, the wound management system 114 may determine the actions 132 based on the probabilities predicted by the machine learning model 126. For example, the wound management system 114 may use the predicted probabilities and the predicted wounds to query a database or repository that stores wounds and corresponding remedial actions 132. The database or repository may then return the remedial actions 132. The wound management system 114 communicates the determined actions 132 to the patient 102 and the healthcare facility 110. For example, the action 132 may include a healthcare facility recommendation and a treatment or recovery plan. The wound management system may communicate the healthcare facility recommendation to the device 104 of the patient 102. The patient 102 may read the healthcare facility recommendation and decide to visit that recommended healthcare facility. The wound management system 114 may communicate the recovery or treatment plan to the healthcare facility 110. The healthcare facility 110 may then implement that treatment or recovery plan to reduce the likelihood that the patient 102 will sustain particular wounds while visiting the healthcare facility 110.

In certain embodiments, the wound management system 114 communicates the action 132 to the patient 102 or the healthcare facility 110 in response to a determination that the action 132 reduces the likelihood that the patient 102 will sustain a particular wound while visiting the healthcare facility 110 by an amount greater than a threshold 802. Stated differently, if the wound management system 114 determines that the action 132 reduces the predicted likelihood that the patient 102 will sustain a particular wound while visiting the healthcare facility 110 by an amount greater than the threshold 802, the wound management system 114 communicates the action 132 to the patient 102 and the healthcare facility 110. If the wound management system 114 determines that the action 132 will not reduce the likelihood that the patient 102 will sustain the particular wound while visiting the healthcare facility 110 by an amount that exceeds the threshold 802, the wound management system 114 may not communicate the action 132 to the patient 102 or the healthcare facility 110. If the threshold 802 is set to zero percent, then all actions 132 that reduce the likelihood by any amount will be presented to the patient 102 or the healthcare facility 110. By setting the threshold 802 to be a larger value (e.g., one percent or two percent), certain actions 132 may be filtered out. As a result, the patient 102 and the healthcare facility 110 can focus on implementing actions 132 that are predicted to provide better results for the patient 102.

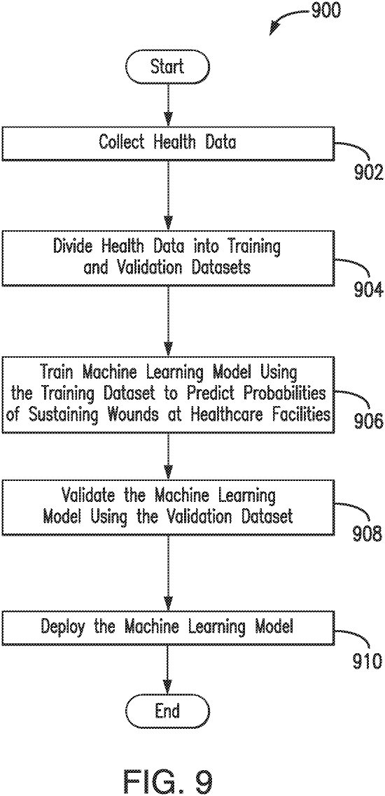

FIG. 9 is a flow chart of an example method 900 performed in the system 100 of FIG. 1. In particular embodiments, the wound management system 114 performs the method 900. By performing the method 900, the wound management system 114 trains a machine learning model 126 to predict likelihoods that a patient 102 will sustain particular wounds while visiting a healthcare facility.

In block 902, the wound management system 114 collects health data from patients 202. Healthcare facilities that are treating the patients 202 may record information about the patients 202, the wounds sustained by the patients 202, and the treatment or remedy applied to the patients 202. The health data may include information that identifies the patients 202 (e.g., names and addresses), the healthcare facilities (e.g., names and addresses), the wounds sustained, the dates on which the wounds were sustained, and the treatments or remedies applied.

In block 904, the wound management system 114 divides the health data into training data 204 and validation data 206. The wound management system 114 may divide the health data in any suitable manner. For example, the wound management system 114 may divide the health data randomly into the training data 204 and the validation data 206. As another example, the wound management system 114 may select the datapoints in the health data that are most different from each other to form the training data 204. In some embodiments, the wound management system 114 clusters the datapoints in the health data such that the datapoints that are most similar to each other are assigned to the same cluster. The wound management system 114 then selects datapoints from different clusters to form the training data 204. As a result, the training data 204 includes diverse datapoints, which improves the accuracy and robustness of the machine learning model that is trained using the training data set 204. The datapoints remaining in the health data after forming the training data 204 may be included in the validation data 206.

In block 906, the wound management system 114 trains the machine learning model 126 using the training data 204 to predict probabilities of sustaining wounds at healthcare facilities. For example, the wound management system 114 may have the machine learning model 126 detect patterns and trends within the training data 204 and learn the wounds sustained when those detected patterns or trends exist. In this manner, the machine learning model 126 is trained to predict the likelihood that certain wounds are sustained when certain patterns or trends are detected.

In block 908, the wound management system 114 validates the machine learning model using the validation data 206. For example, the wound management system 114 may apply the machine learning model 126 to datapoints in the validation data 206 to predict the likelihood of certain wounds being sustained given the datapoints in the validation data 206. The wound management system 114 may then compare the predicted likelihood with the actual wounds sustained indicated by the validation data 206. This comparison indicates a loss or accuracy of the machine learning model. When the loss or accuracy of the machine learning model is at an acceptable level, the wound management system 114 considers the machine learning model 126 trained. If the loss or accuracy of the machine learning model 126 is not at an acceptable level, the wound management system 114 may perform additional rounds of training for the machine learning model 126.

In block 910, the wound management system 114 deploys the machine learning model 126. In some embodiments, the wound management system 114 deploys the machine learning model 126 so that the wound management system 114 may use the machine learning model 126 itself. In other embodiments, the wound management system 114 deploys the machine learning model 126 to a device 104 of the patient 102. In these embodiments, the wound management system 114 may be embodied within the device 104 and the device 104 may apply the machine learning model 126 to health screening data 124 provided by the patient 102.

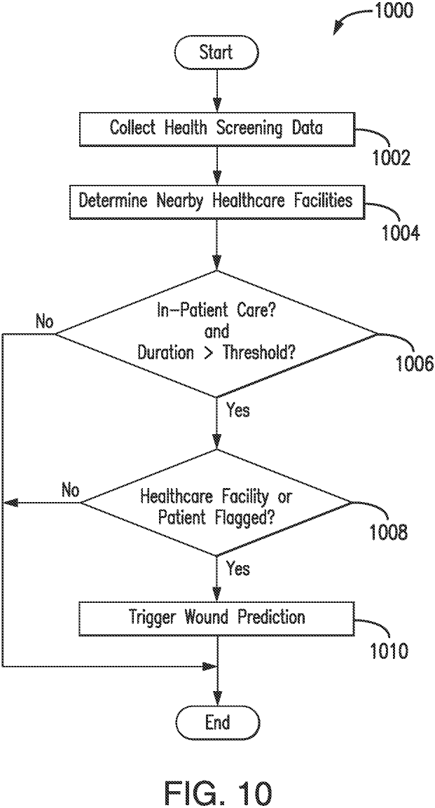

FIG. 10 is a flowchart of an example method 1000 performed in the system 100 of FIG. 1. In particular embodiments, the wound management system 114 performs the method 1000. By performing the method 1000 the wound management system 114 determines whether certain trigger conditions have been met for applying the machine learning model 126.

In block 1002, the wound management system 114 collects health screening data 124 from a patient 102. The patient 102 may provide the health screening data 124 using the device 104. The device 104 may communicate the health screening data 124 to the wound management system 114. The health screening data 124 may include information about the patient 102. For example, the health screening data 124 may include information that identifies the patient 102, such as a name or address. As another example, the health screening data may include medical information of the patient 102, such as symptoms experienced by the patient 102 or allergies of the patient 102. The health screening data 124 may also include medications and vaccinations taken by the patient 102 as well as previous hospitalizations and operations of the patient 102. The health screening data 124 may also include information about the lifestyle of the patient 102. For example, the health screening data 124 may indicate the home conditions, work conditions, and activities or events in which the patient 102 engages. The health screening data 124 may also include indications of whether the patient 102 smokes, drinks, wears a seatbelt, or uses recreational drugs.

In block 1004, the wound management system 114 determines healthcare facilities that are near the patient 102. For example, the wound management system 114 may determine the healthcare facilities that are within a certain distance of the address provided by the patient 102 in the health screening data 124 or the healthcare facilities that are indicated by the patient 102 as preferred facilities. The wound management system 114 may consider the healthcare facilities that are proximate to the patient 102 as candidate healthcare facilities for the patient 102.

In block 1006, the wound management system 114 determines whether in-patient care is needed and whether the duration of the care exceeds a threshold. The wound management system 114 may determine whether in-patient care is needed by analyzing the symptoms experienced by the patient 102 as provided in the health screening data 124. For example, if the symptoms are severe or if the symptoms greatly impact the health of the patient 102, then the wound management system 114 may determine that in-patient care is needed. Additionally, the wound management system 114 may determine a duration of that in-patient care. If that duration exceeds a threshold 514, then the wound management system 114 may proceed. If in-patient care is not needed or if the duration of the care does not exceed the threshold 514, then the wound management system 114 may conclude the method 1000.

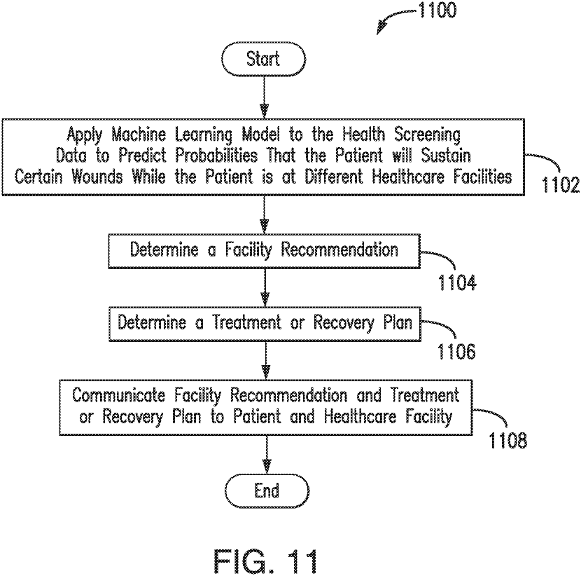

In block 1008, the wound management system 114 determines whether a candidate healthcare facility or the patient 102 is flagged for wounds previously sustained. For example, if a candidate healthcare facility has a history of patients sustaining wounds while visiting that healthcare facility, then the healthcare facility may be flagged. If the patient 102 has previously sustained wounds while visiting a healthcare facility, then the patient 102 may be flagged. If the candidate healthcare facilities and the patient 102 are not flagged, then the wound management system 114 may conclude the method 1000. If a candidate healthcare facility or the patient 102 are flagged, then the wound management system 114 may continue to block 1010 to trigger wound prediction. When wound prediction is triggered, the wound management system 114 determines that the machine learning model 126 should be applied to the health screening data 124 provided by the patient 102. The wound management system 114 may proceed to the method shown in FIG. 11 to apply the machine learning model 126.

FIG. 11 is a flow chart of an example method 1100 performed in the system 100 of FIG. 1. In particular embodiments, the wound management system 114 performs the method 1100. By performing the method 1100, the wound management system 114 predicts likelihoods that a patient 102 will sustain particular wounds while visiting a healthcare facility.

In block 1102, the wound management system 114 applies the machine learning model 126 to the health screening data 124 to predict probabilities that the patient 102 will sustain certain wounds while the patient 102 is visiting different healthcare facilities. The machine learning model 126 may compare the health screening data 124 to the previous patient data 116 that indicated the wounds sustained by previous patients 202 at different healthcare facilities. The machine learning model then determines whether certain patterns or trends in the health screening data 124 match or are similar to patterns or trends in the previous patient data 116 for patients 202 at a particular healthcare facility. Based on whether these patients 202 sustained certain wounds while they were at this healthcare facility, the machine learning model may determine the likelihood that the patient 102 will sustain certain wounds if the patient 102 were to visit this healthcare facility. For example, the machine learning model 126 may predict, based on the health screening data 124, that the patient is likely to experience bedsores if the patient 102 visits a particular healthcare facility.

In block 1104, the wound management system 114 determines a facility recommendation based on the predicted probabilities. For example, the wound management system 114 may recommend a healthcare facility for which the wound management system has predicted lower probabilities of wounds being sustained while the patient 102 is visiting that healthcare facility. As another example, the wound management system 114 may recommend a healthcare facility for which the machine learning model 126 predicted a lower probability that the patient 102 will sustain a wound that the patient 102 had previously sustained at another healthcare facility.

In block 1106, the wound management system 114 determines a treatment or recovery plan for the predicted wounds. For example, if the wound management system 114 predicts that the patient 102 is likely to experience bedsores when visiting a healthcare facility, the wound management system 114 may determine that the patient 102 should be moved frequently and the patient’s 102 bed sheets should be changed regularly as part of the treatment or recovery plan. The wound management system 114 may query a database or repository of stored treatment or recover plans using the predicted wounds. The database or repository then returns the treatment or recovery plans that are tied to the predicted wounds.

In block 1108, the wound management system 114 communicates the facility recommendation and treatment or recovery plan to the patient 102 and the healthcare facility. For example, the wound management system 114 may communicate the facility recommendation to the patient 102 to let the patient 102 know which healthcare facility the patient 102 should visit to reduce a likelihood that the patient 102 will sustain a particular wound while visiting that healthcare facility. As another example, the wound management system 114 may communicate the treatment or recovery plan to the healthcare facility to reduce the likelihood that the patient 102 will sustain the wound while visiting that healthcare facility. In this manner, the wound management system 114 improves the health and safety of the patient 102 and reduces the number of incidences or occurrences of wounds while patients are visiting the healthcare facility.

In certain embodiments, by performing the methods 1000 and 1100, the wound management system 110 can predict the likelihood that a patient 102 will sustain a wound in particular healthcare facilities. Specifically, patients 102 often relied on word of mouth and anecdotes to assess the likelihood that the patients 102 would sustain wounds while the patients 102 were at certain healthcare facilities. Therefore, the patients 102 were not performing an accurate assessment of their likelihood of sustaining wounds, which did not improve the health and well-being of the patients 102. Additionally, the healthcare facilities did not assess the likelihood of the patients 102 sustaining wounds. Rather, the healthcare facilities merely treated wounds as they were sustained. Therefore, the incidences and occurrences of wounds was not decreasing.

The wound management system 114, on the other hand, uses machine learning to assess the likelihood of a patient 102 sustaining a wound at certain healthcare facilities (e.g., healthcare facilities 110 and 112). As a result, the wound management system 114 provides the technical advantage of a more accurate prediction of the likelihood that the patient 102 will sustain a wound at the healthcare facilities, in certain embodiments. The wound management system 114 also provides recommendations to the patient 102 (e.g., a healthcare facility recommendation) and to the healthcare facilities (e.g., a treatment or remedy plan recommendation) based on these more accurate predictions, which effects a particular treatment or prophylaxis for preventing or reducing the likelihood of sustaining wounds at the healthcare facilities.

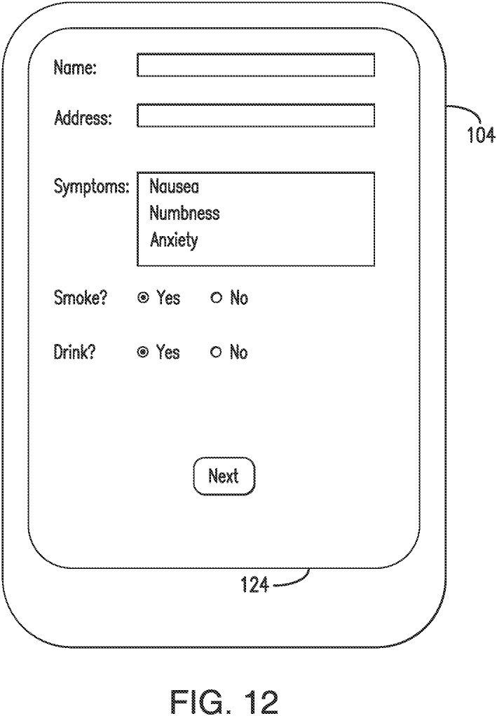

FIG. 12 illustrates an example device 104 in the system 100 of FIG. 1. As seen in FIG. 12, the device 104 presents an interface that the patient 102 may use to provide health screening data 124. The interface shown in FIG. 12 includes fields that the patient 102 may use to enter information that identifies the patient 102, such as a name or address. Additionally the interface includes a field in which the patient 102 may select or enter symptoms experienced by the patient 102. In the example of FIG. 12, the patient 102 has indicated that the patient 102 is experiencing nausea, numbness, and anxiety. Additionally, the interface includes fields that the patient 102 may use to indicate lifestyle or habits of the patient 102. In the example of FIG. 12, the interface includes fields that the patient 102 has used to indicate that the patient 102 smokes and drinks. After the patient 102 has provided the health screening data 124, the device 104 communicates the health screening data 124 to the wound management system 114.

The wound management system 114 may analyze the information in the health screening data 124 to predict likelihoods that the patient 102 will sustain wounds while visiting a healthcare facility. Based on these predicted likelihoods, the wound management system may provide healthcare facility recommendations and recovery or treatment plans that reduce the likelihood that the patient 102 will sustain these wounds. The wound management system 114 then provides the healthcare facility recommendation and the recovery or treatment plan to the patient 102 and to the healthcare facility.

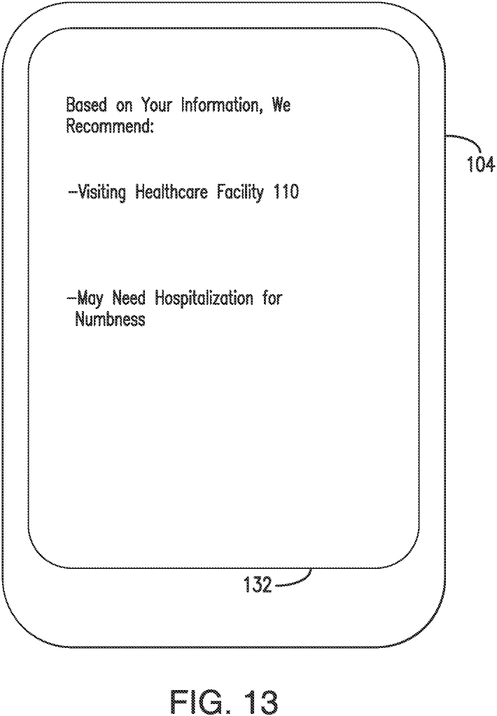

FIG. 13 illustrates an example device 104 in the system 100 of FIG. 1. As seen in FIG. 13, the device 104 presents a message from the wound management system 114 that includes an action 132. The wound management system 114 may have provided this message to a patient 102 to recommend a healthcare facility and to let the patient 102 know of what to expect at the healthcare facility. In the example of FIG. 13, the wound management system 114 recommends that the patient 102 visit the healthcare facility 110 for the symptoms experienced by the patient 102. Additionally, the wound management system 114 alerts the patient 102 that the patient 102 may need hospitalization for the numbness experienced by the patient 102. The wound management system 114 may have determined that the numbness may require in-patient care for a duration that exceeds a threshold. In response, the wound management system 114 includes in the message an alert for the patient 102 that the patient 102 may need hospitalization for the numbness.

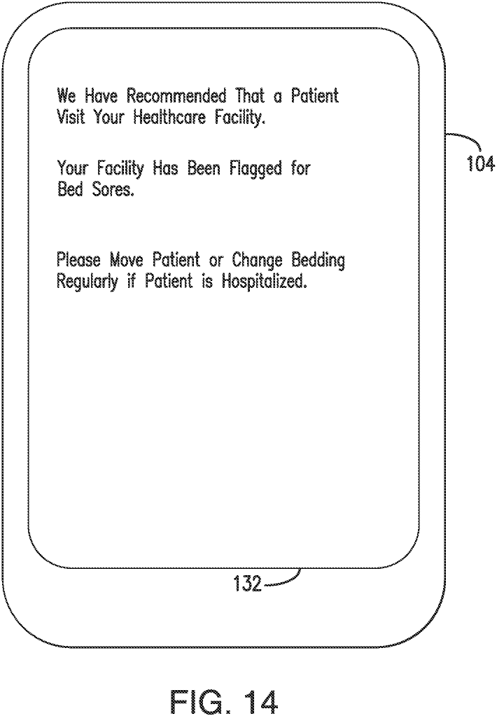

FIG. 14 illustrates an example device 104 in the system 100 of FIG. 1. The device 104 may belong to an operator or a caregiver at the healthcare facility. The device 104 displays a message from the wound management system 114. The message may include an action 132 determined by the wound management system 114 based on the predicted probabilities that the patient 102 will sustain particular wounds while visiting the healthcare facility. In the example of FIG. 14, the message indicates that the wound management system 114 has recommended that the patient 102 visit the healthcare facility. Additionally, the message indicates that the healthcare facility has been previously flagged for bedsores. The message then provides the remedial action 132 of moving the patient 102 or changing the patient’s 102 bedding regularly if the patient 102 is hospitalized. The healthcare facility may then perform these remedial actions 132 to reduce the likelihood that the patient 102 will experience bedsores while visiting the healthcare facility.

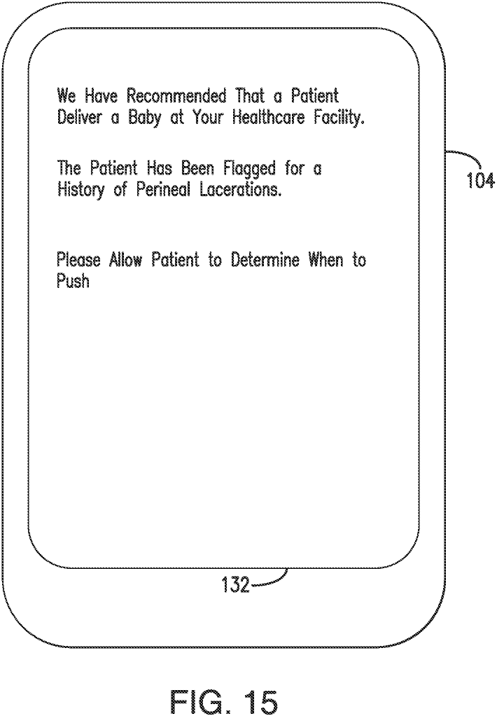

FIG. 15 illustrates an example device 104 in the system 100 of FIG. 1. The device 104 may belong to an operator or a caregiver in the healthcare facility. As seen in FIG. 15, the device 104 displays a message from the wound management system 114. The message indicates that the wound management system 114 has recommended that a patient 102 deliver a baby at the healthcare facility. The message also indicates that the healthcare facility has been previously flagged for a history of perineal lacerations. The message includes a remedial action 132 that the healthcare facility may perform or take to reduce the likelihood that the patient 102 will sustain a perineal laceration while visiting the healthcare facility. Specifically, the message recommends that the healthcare facility allow the patient 102 to determine when to start and stop pushing, which may reduce the likelihood of developing perineal lacerations. As a result the wound management system 114 reduces the probability that the patient 102 will sustain a particular wound while visiting the healthcare facility in particular embodiments.

In summary, a wound management system 114 uses machine learning to predict whether a patient 102 is likely to sustain different wounds at different healthcare facilities 110 or 112 based on information about the patient 102. For example, the wound management system 114 may predict that a patient 102 is more likely to sustain particular wounds at one healthcare facility 110 relative to another healthcare facility 112. In response, the wound management system 114 may recommend that the patient 102 visit one healthcare facility 110 over the other. As another example, the wound management system 114 may predict that a patient 102 has an unacceptably high likelihood of sustaining a particular wound at a healthcare facility 110. In response, the wound management system 114 may recommend that certain remedial actions 132 be taken by the healthcare facility 110 to prevent the wound from occurring. In this manner, the wound management system 114 provides a proactive approach towards wound treatment, which improves the health and well-being of the patient 102, in certain embodiments.

### Example Clauses

Implementation examples are described in the following numbered clauses:

Clause 1: A method includes collecting data relating to a patient’s health comprising symptoms experienced by the patient and in response to determining that the symptoms experienced by the patient necessitate in-patient care, applying a machine learning model to the data relating to the patient’s health to predict a first probability that the patient will sustain a first wound while the patient is at a first healthcare facility. The method also includes, in response to determining, based on the first probability, that the patient should be treated at the first healthcare facility, determining an action that reduces the first probability that the patient will sustain the first wound and communicating, to the first healthcare facility, a message indicating that the action should be taken to reduce the first probability that the patient will sustain the first wound while the patient is at the first healthcare facility.

Clause 2: The method of Clause 1, further including determining a second probability that the patient will sustain the first wound while the person is at a second healthcare facility different from the first healthcare facility. Determining that the patient should be treated at the first healthcare facility may include comparing the first probability to the second probability.

Clause 3: The method of any of Clauses 1-2, further including applying the machine learning model to the data relating to the patient’s health to predict a second probability that the patient will sustain a second wound while the patient is at the first healthcare facility. Determining that the patient should be treated at the first healthcare facility may be further based on the second probability.

Clause 4: The method of any of Clauses 1-3, further including collecting a dataset indicating (i) a plurality of past physical wounds sustained by different patients while the different patients were in the first healthcare facility and (ii) a plurality of wound types of the plurality of past physical wounds, training the machine learning model using the training dataset, and validating the machine learning model using the validation dataset after the machine learning model is trained.

Clause 5: The method of any of Clauses 1-4, further including removing portions of the dataset that were generated prior to a time threshold before training the machine learning model.

Clause 6: The method of any of Clauses 1-5, wherein applying the machine learning model to the data relating to the patient’s health is further in response to determining that the first healthcare facility is within a distance threshold of the patient and that the first healthcare facility is flagged for previous wounds sustained at the first healthcare facility.

Clause 7: The method of any of Clauses 1-6, wherein applying the machine learning model to the data relating to the patient’s health is further in response to determining that the patient is flagged for previous wounds sustained by the patient.

Clause 8: The method of any of Clauses 1-7, wherein applying the machine learning model to the data relating to the patient’s health is further in response to determining that the in-patient care is predicted to last for a duration that exceeds a threshold.

Clause 9: The method of any of Clauses 1-8, wherein communicating the message is in response to determining that the action reduces the first probability by an amount exceeding a threshold.

Clause 10: An apparatus including a memory and a hardware processor communicatively coupled to the memory configured to perform a method in accordance with any one of Clauses 1-9.

Clause 11: A non-transitory computer-readable medium including instructions that, when executed by a processor, cause the processor to perform a method in accordance with any one of Clauses 1-9.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

In the preceding, reference is made to embodiments presented in this disclosure. However, the scope of the present disclosure is not limited to specific described embodiments. Instead, any combination of the described features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the preceding aspects, features, embodiments and advantages are merely illustrative and are not considered elements or limitations of the appended claims except where explicitly recited in a claim(s).

As will be appreciated by one skilled in the art, the embodiments disclosed herein may be embodied as a system, method or computer program product. Accordingly, aspects may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects may take the form of a computer program product embodied in one or more computer readable medium(s) having computer readable program code embodied thereon.

Any combination of one or more computer readable medium(s) may be utilized. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium is any tangible medium that can contain, or store a program for use by or in connection with an instruction execution system, apparatus or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider).

Aspects of the present disclosure are described below with reference to flowchart illustrations and/or block diagrams of methods, apparatus (systems) and computer program products according to embodiments presented in this disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality and operation of possible implementations of systems, methods and computer program products according to various embodiments. In this regard, each block in the flowchart or block diagrams may represent a module, segment or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

What is claimed is:

1. A method comprising:
collecting data relating to a patient’s health comprising symptoms experienced by the patient;
in response to determining that the symptoms experienced by the patient necessitate in-patient care, applying a machine learning model to the data relating to the patient’s health to predict a first probability that the patient will sustain a first wound while the patient is at a first healthcare facility;
in response to determining, based on the first probability, that the patient should be treated at the first healthcare facility, determining an action that reduces the first probability that the patient will sustain the first wound; and
communicating, to the first healthcare facility, a message indicating that the action should be taken to reduce the first probability that the patient will sustain the first wound while the patient is at the first healthcare facility.

2. The method of claim 1, further comprising:
determining a second probability that the patient will sustain the first wound while the patient is at a second healthcare facility different from the first healthcare facility,
wherein determining that the patient should be treated at the first healthcare facility comprises comparing the first probability to the second probability.

3. The method of claim 1, further comprising:
applying the machine learning model to the data relating to the patient’s health to predict a second probability that the patient will sustain a second wound while the patient is at the first healthcare facility,
wherein determining that the patient should be treated at the first healthcare facility is further based on the second probability.

4. The method of claim 1, further comprising:
collecting a dataset indicating (i) a plurality of past physical wounds sustained by different patients while the different patients were in the first healthcare facility and (ii) a plurality of wound types of the plurality of past physical wounds;
dividing the dataset into a training dataset and a validation dataset;
training the machine learning model using the training dataset; and
validating the machine learning model using the validation dataset after the machine learning model is trained.

5. The method of claim 4, further comprising:
removing portions of the dataset that were generated prior to a time threshold before training the machine learning model.

6. The method of claim 1, wherein:
applying the machine learning model to the data relating to the patient’s health is further in response to determining that the first healthcare facility is within a distance threshold of the patient and that the first healthcare facility is flagged for previous wounds sustained at the first healthcare facility.

7. The method of claim 1, wherein:
applying the machine learning model to the data relating to the patient’s health is further in response to determining that the patient is flagged for previous wounds sustained by the patient.

8. The method of claim 1, wherein:
applying the machine learning model to the data relating to the patient’s health is further in response to determining that the in-patient care is predicted to last for a duration that exceeds a threshold.

9. The method of claim 1, wherein:
communicating the message is in response to determining that the action reduces the first probability by an amount exceeding a threshold.

10. An apparatus comprising:
a memory; and
a hardware processor communicatively coupled to the memory, the hardware processor configured to:
collect data relating to a patient’s health comprising symptoms experienced by the patient;
in response to determining that the symptoms experienced by the patient necessitate in-patient care, apply a machine learning model to the data relating to the patient’s health to predict a first probability that the patient will sustain a first wound while the patient is at a first healthcare facility;
in response to determining, based on the first probability, that the patient should be treated at the first healthcare facility, determine an action that reduces the first probability that the patient will sustain the first wound; and
communicate, to the first healthcare facility, a message indicating that the action should be taken to reduce the first probability that the patient will sustain the first wound while the patient is at the first healthcare facility.

11. The apparatus of claim 10, wherein the hardware processor is further configured to:
determine a second probability that the patient will sustain the first wound while the patient is at a second healthcare facility different from the first healthcare facility,
wherein determining that the patient should be treated at the first healthcare facility comprises comparing the first probability to the second probability.

12. The apparatus of claim 10, wherein the hardware processor is further configured to:
apply the machine learning model to the data relating to the patient’s health to predict a second probability that the patient will sustain a second wound while the patient is at the first healthcare facility,
wherein determining that the patient should be treated at the first healthcare facility is further based on the second probability.

13. The apparatus of claim 10, wherein the hardware processor is further configured to:
collect a dataset indicating (i) a plurality of past physical wounds sustained by different patients while the different patients were in the first healthcare facility and (ii) a plurality of wound types of the plurality of past physical wounds;
divide the dataset into a training dataset and a validation dataset;
train the machine learning model using the training dataset; and
validate the machine learning model using the validation dataset after the machine learning model is trained.

14. The apparatus of claim 13, wherein the hardware processor is further configured to:
remove portions of the dataset that were generated prior to a time threshold before training the machine learning model.

15. The apparatus of claim 10, wherein:
applying the machine learning model to the data relating to the patient’s health is further in response to determining that the first healthcare facility is within a distance threshold of the patient and that the first healthcare facility is flagged for previous wounds sustained at the first healthcare facility.

16. The apparatus of claim 10, wherein:
applying the machine learning model to the data relating to the patient’s health is further in response to determining that the patient is flagged for previous wounds sustained by the patient.

17. The apparatus of claim 10, wherein:
applying the machine learning model to the data relating to the patient’s health is further in response to determining that the in-patient care is predicted to last for a duration that exceeds a threshold.

18. The apparatus of claim 10, wherein:
communicating the message is in response to determining that the action reduces the first probability by an amount exceeding a threshold.

19. A non-transitory computer readable medium storing instructions that, when executed by a processor, cause the processor to:
collect data relating to a patient’s health comprising symptoms experienced by the patient;
in response to determining that the symptoms experienced by the patient necessitate in-patient care, apply a machine learning model to the data relating to the patient’s health to predict a first probability that the patient will sustain a first wound while the patient is at a first healthcare facility;
in response to determining, based on the first probability, that the patient should be treated at the first healthcare facility, determine an action that reduces the first probability that the patient will sustain the first wound; and
communicate, to the first healthcare facility, a message indicating that the action should be taken to reduce the first probability that the patient will sustain the first wound while the patient is at the first healthcare facility.

20. The non-transitory computer readable medium of claim 19, wherein:
applying the machine learning model to the data relating to the patient’s health is further in response to determining that the first healthcare facility is within a distance threshold of the patient and that the first healthcare facility is flagged for previous wounds sustained at the first healthcare facility.
